SEQUENCE LISTING (13 sequences)

SEQ ID NO: 1 - protein, 422 aa, synthetic construct
VPGVGVPGAGVPGVGVPGVGVPGVGVPGVGVPGAGVPGVGVPGVGVPGGKGVGVPGVGVGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGY

SEQ ID NO: 2 - protein, 424 aa, synthetic construct
VPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKGVPGXGVPGXGVPGXGVPGXGVPGXGVPGXGVPGXGVPGXGVPGXGVPGXGGKG

SEQ ID NO: 3 - protein, 5 aa, synthetic construct
VPGXG

SEQ ID NO: 4 - protein, 200 aa, synthetic construct
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 5 - protein, 231 aa, synthetic construct
MSKGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGSTNPKPQRKTKRNTNRRPQDVKFPGGGQIV

SEQ ID NO: 6 - protein, 564 aa, synthetic construct
MGVGVPGVGVPGAGVPGVGVPGVGVPGVGVPGVGVPGAGVPGVGVPGVGVPGGKGVGVPGVGVGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGYNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQR

SEQ ID NO: 7 - protein, 608 aa, synthetic construct
MSKGPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGWP

SEQ ID NO: 8 - protein, 505 aa, synthetic construct
MGHGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGW

SEQ ID NO: 9 - protein, 605 aa, synthetic construct
MGHGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGVGVPGHGVPGGGVPGHGVPGAGVPGW

SEQ ID NO: 10 - protein, 424 aa, synthetic construct
VPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHGVPGXGVPGXGVPGXGVPGXGVPGXGVPGXGVPGXGVPGXGVPGXGVPGXGGHG

SEQ ID NO: 11 - protein, 124 aa, synthetic construct
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVID

SEQ ID NO: 12 - DNA, 28 bp, synthetic construct
  source 1..28: mol_type = other DNA
tttttcctagagagtagagcctgcttcg

SEQ ID NO: 13 - DNA, 28 bp, synthetic construct
  source 1..28: mol_type = other DNA
tttttcctagagagtagagcctgcttcg

(19) United States
(12) Patent Application Publication
(10) Pub. No.: US 2023/0407367 A1
(43) Pub. Date: Dec. 21, 2023

(54) INTRINSICALLY DISORDERED PROTEINS FOR EXTRACTION OF NUCLEIC ACIDS

(71) Applicants: Gabriel P. Lopez, Penasco, NM (US); Nichlaus J. Carroll, Albuquerque, NM (US); Telmo Diez Perez, Albuquerque, NM (US); David S. Peabody, Albuquerque, NM (US)

(72) Inventors: Gabriel P. Lopez, Penasco, NM (US); Nichlaus J. Carroll, Albuquerque, NM (US); Telmo Diez Perez, Albuquerque, NM (US); David S. Peabody, Albuquerque, NM (US)

(21) Appl. No.: 18/137,921

(22) Filed: Apr. 21, 2023

Related U.S. Application Data
(60) Provisional application No. 63/337,874, filed on May 3, 2022.

(51) Int. Cl. C12Q 1/6806

(52) U.S. Cl. CPC C12Q 1/6806

(57) ABSTRACT

The present disclosure relates to compositions and methods for isolating nucleic acids from a sample comprising nucleic acids, such as physiologically relevant samples or environmental samples. The sample can be incubated with a population of polymers that bind the nucleic acids to form a coacervate in a liquid-liquid phase separated solution. Incubation can be performed at a temperature above a concentration dependent phase separation transition temperature of the polymers. The resulting coacervate can be decanted from the liquid-liquid phase separated solution. The nucleic acids can be separated from the polymers by adding a salt solution, adjusting the pH, or both to disrupt an electrostatic interaction between the nucleic acids and the polymers.

### CROSS-REFERENCE TO RELATED APPLICATION

This application claims the priority of U.S. provisional application Ser. No. 63/337,874, filed May 3, 2022, the disclosure of which is incorporated herein by reference in its entirety as if fully set forth herein.

### FEDERAL FUNDING

This technology was made with government support under CBET-2031774, CBET-2048051, and MCB-2123465 from the National Science Foundation. The government has certain rights in the technology.

### INCORPORATION BY REFERENCE OF SEQUENCE LISTING

A Sequence Listing is provided herewith as an xml file, “2346033.xml” created on Jun. 23, 2023 and having a size of 16,437 bytes. The content of the xml file is incorporated by reference herein in its entirety.

### BACKGROUND OF THE TECHNOLOGY

The separation of nucleic acids (NAs), such as DNA and RNA, from biological samples, known as NA extraction or isolation, is a first step in many analytical, diagnostic, molecular biological, and forensic procedures. The NA isolation process typically involves several steps, including inactivation of resident nucleases to preserve NA integrity, cellular disruption, separation of the NA from cellular contaminants, and concentration of the extracted NA for further analysis. Currently used processes can be categorized into two general types of extraction methods. One example is liquid-liquid extraction by guanidium thiocyanate-phenol-chloroform extraction. Another example is solid-phase extraction include use silica-based, microchromatographic columns (e.g., “spin columns”) or charged magnetic beads.

There is no universally established standardized technique for NA extraction to use in multiple application contexts. Available techniques require varying degrees of processing time, instrumentation, use of hazardous reagents, trained personnel, and well-maintained laboratory spaces, each providing potential impediments to implementation in low-resource settings and miniaturized point-of-care devices.

### SUMMARY

As described herein, NA extraction from biologically relevant solutions can be performed using triggered liquid-liquid phase separation of NA-binding intrinsically disordered proteins (IDPs). Two types of NA-binding IDPs are provided and are based on genetically engineered elastin-like polypeptides (ELPs). ELPs are model IDPs that exhibit a lower critical solution temperature in water and can be designed to exhibit liquid-liquid phase separation (LLPS) at desired temperatures in a variety of biological solutions. ELP fusion proteins with NA-binding domains can be used to extract DNA and RNA from biological solutions. LLPS of pH responsive ELPs that incorporate histidine in their amino acid sequences can be used for binding, extraction, and release of NAs from biological solutions such as for detection of SARS-CoV-2 RNA in samples from COVID+ patients.

### BRIEF DESCRIPTION OF THE DRAWINGS

Aspects of the present disclosure can be better understood with reference to the following drawings.

FIG. 1 is a schematic illustrating the formation of E3 coacervate and capture of DNA.

FIG. 2 is a graph depicting Characterization of E3 Tt(ϕ) in the absence and presence of 0.5 μM ssDNA.

FIG. 3 depicts fluorescence microscopy images of phase and ssDNA-Cy3 partitioning behavior of 1 mM E3 within aqueous microdrops in oil: (Panels A-C) With added 100 mM NaCl and (Panels D-F) with no added NaCl. (Panels A1-F1) Brightfield microscopy shows thermally induced spinodal decomposition of E3 (1 mM initial concentration) into fully coarsened condensate spheres, with similar phase behavior for both + and —NaCl solutions. (Panels A2-F2) Time lapse fluorescence microscopy of ssDNA-Cy3 (0.5 μM). Above the E3 Tt (T=55° C.) and without added NaCl, the fluorescent DNA is efficiently sequestered in E3 condensates. Adding NaCl results in electrostatic shielding leaving much more of DNA in the solvent-rich phase. (Panels A3-F3) Overlay of time lapse brightfield and fluorescence microscopy images depicting E3 phase separation and concurrent ssDNA-Cy3 localization in microdrops at T=55° C. Scale bars=50 μm.

FIGS. 4A-4D are graphs depicting salt mediated DNA capture by E3 coacervates at T=55° C. Experimental data (marker points with standard deviation) of (FIG. 4A) dilute phase DNA concentration fraction from initial (C′/C0) DNA and (FIG. 4B) E3 coacervate volume fraction in microdrops used as FH fitting parameters (solid curves are interpolations of the tie lines from resulting FH phase diagrams). FIG. 4C shows dependence on ionic strength for Debye-Hückel modified FH interaction parameters (lines) and experimental FH interaction parameters (marker points). FIG. 4D shows ternary phase diagrams comprising binodal curves and a few representative tie lines for E3, DNA and Buffer for added salt concentrations of 0 mM NaCl (solid lines) and 100 mM NaCl (dashed lines). Significant points are marked as circles.

FIGS. 5A-5B depict a schematic and a graph demonstrating a two-step DNA purification method. FIG. 5A illustrates a workflow for the two-step DNA purification method. FIG. 5B is a graph depicting the results of fluorimetry measurements takes after Step 1 (left side) and after Step 2 (right side). E2-488 fluorescence is shown as light circles and the blank bars. DNA-Cy3 fluorescence is shown as dark circles and the spotted bars. Each circle represents a measurement of an individual sample. The bars show the average of the circular data points and the error bars show the standard deviation.

FIG. 6 is a schematic illustrating how engineered IDPs can isolate viral RNA by phase separation in complex samples for viral RNA for detection and diagnosis.

FIG. 7 is a schematic illustrating an IDP of SEQ ID NO. 1 (shown as “E3”) followed by an 87 amino acid RNA recognition motif (“RRM”) from FUS protein which comprises an RNA binding folded domain.

FIG. 8 is a graph depicting The RNA binding profile of COR 124 as a function of concentration.

FIGS. 9A-9C depict the recruitment of ssDNA and tRNA into ELP coacervates upon LLPS at 50° C. in different biologically relevant fluids. FIG. 9A is a schematic illustration of a workflow for examining recruitment of NAs into ELP coacervates upon LLPS in different media. To liberate the NAs from the ELPs prior to running the gels, samples were incubated in a stopping buffer. 2.5% agarose gels were stained with SyBr Gold to illustrate the recruitment of 0.1 mg/ml tRNA (FIG. 9B) and 0.5 μM ssDNA (FIG. 9C) into the protein rich phase (PRP) of 0.1 mM E1.40COR30 and 0.1 mM E3.10 upon LLPS.

FIG. 10 is a schematic illustrating a workflow for quantifying SARS-CoV-2 RNA in complex coacervates using qPCR. The workflow quantifies the number of viral RNA copies that phase separate within the ELP coacervates. After LLPS, coacervates are resuspended in viral transfer media (VTM) and RNA is separated from protein using a commercial chromatographic method prior to RT-qPCR.

FIGS. 11A-11B show the pH-sensitive charge and LCST behavior of H-20 and H-24. FIG. 11A is a graph of the estimation by SnapGene of molecular charge vs pH for H-20 and H-24. FIG. 11B is a graph showing the cloud point transition temperature (Tt) for LLPS of 0.5 mM H-20 and H-24. Tt was measured in the absence and presence of 0.5 μM ssDNA in pH 6 buffer (37 mM citric acid/126 mM Na2HPO4) and pH 9 buffer (100 mM Tris).

FIG. 12 are images of agarose gels showing binding of His-ELPs and NAs at pH 6. 2.5% agarose gels stained with SyBr Gold illustrate the concentration dependent binding activity of H-20 and H-24, but not of E3 with 0.5 μM ssDNA (gels A1-A3) and 0.5 mg/ml tRNA (gels B1-B3) in 37 mM citric acid/126 mM Na₂HPO₄ buffer at pH 6. (H-20 and H-24 concentrations are: 0.1, 1, 5, 10, 25, 50 and 100 μM. E3 concentrations are: 10, 100, 1000 PM.)

FIG. 13 is an image of an agarose gel showing lack of binding of His-ELPs at basic pH 9 (4° C.). 2.5% agarose gels stained with SyBr Gold illustrate the absence of appreciable binding at 4° C. of 100 μM H-20 and H-24 with 0.5 mg/mL tRNA (lanes 1-3) and 0.5 μM ssDNA (lanes 4-6) in 100 mM Tris buffer at pH 9.

FIGS. 14A-14B are images of agarose gels showing gel retardation assays for H-24 an H-20 His-ELPs binding to ssDNA and tRNA at pH. 8 with 300 mM NaCl. FIG. 14A is an image of a gel showing that the migration of ssDNA and tRNA was not affected by the presence of soluble H-24. FIG. 14B is an image of a gel showing that the migration of ssDNA and tRNA was not affected by the presence of soluble H-20.

FIGS. 15A-15D illustrate recruitment of ssDNA into H-24 coacervate from different physiologically relevant solutions and subsequent release upon LLPS after pH shift. FIG. 15A is a schematic illustration of a workflow of a two-step NA isolation assay. FIG. 15B-15D are graphs depicting fluorimetry measurements of an ATTO488-labeled ssDNA in the supernatant (SN, circles, dark gray bars) and coacervate (squares, light gray bars) taken after LLPS 1 and LLPS 2 for the three physiologically relevant solutions, buffer (FIG. 15B), saliva (FIG. 15C), and a nasal swab (FIG. 15D).

### DETAILED DESCRIPTION

Cellular membraneless organelles (MLOs) are distinct phase separated compartments that lack a lipid membrane but nevertheless function akin to their membrane delineated counterparts via the spatial and temporal organization of molecules. Several MLOs comprise RNA binding intrinsically disordered proteins (IDPs) that undergo reversible liquid-liquid phase separation (LLPS) to assemble and disassemble condensed phase assemblies for a host of regulatory activities. For example, phase separated IDPs bind and sequester cytoplasmic mRNA in MLOs known as stress granules to regulate their activity in response to environmental stresses, sometimes acting with other MLOs such as P-bodies, to regulate mRNA outcome. Examples of environmental stimuli that can lead to rapid assembly and disassembly of IDP coacervate MLOs include temperature, pH, and osmotic stress. Cellular MLOs regulate downstream function using coupled environmental sensing and molecular phase behavior, thus helping to minimize complex, multilevel signaling cascades. Hence, condensed phase cellular MLOs provide a practical blueprint to potentially engineer programmable analogs in synthetic systems. Indeed, the simplicity of this biopolymer solution phase behavior is reflected by gaining popularity of IDPs and MLOs in origin of life discussions.

Further inspiring the use of IDPs in engineered systems are investigations that shed light on the mechanism of protein-NA binding and the role of IDPs in driving cellular MLO assemblies. For example, synthetic nucleoprotein MLOs were assembled in protocells using IDP fusions comprising an elastin-like protein (ELP) block concatenated with a soluble arginine-rich domain (RGG). ELPs are pentameric repeat polymers (sequence VPGXG, X=guest residue) while RGG domains are present in a host of cellular IDPs, including LAF-1, FUS, and MRE11. Relatively hydrophobic ELPs block conferred phase separation behavior to the fusions, while the RGG domain enabled electrostatic binding of the fusions to RNA. In biological systems, RGG domains of cellular IDPs interact with RNA while simultaneously undergoing LLPS and therefore have dual roles as mediators of both RNA-binding phase separation behaviors

The mechanism of dual-role IDPs is further characterized by investigation of synthetic NA-binding IDP surrogates with well-defined stimulus-induced phase behavior that is not driven solely by complexation of IDP and NA polyelectrolytes of opposite charge. In this regard, ELPs are intriguing as candidate surrogates because of their ability to maintain hydrophobic lower critical solution temperature (LCST) phase behavior even while carrying a relatively large mean net charge. Furthermore, the molecular parameters of diverse sets of ELPs (e.g., guest residue, chain length) and their aqueous solubility as a function of temperature, concentration, and presence of cosolutes is correlated.

As described herein, polymers suitable for use in methods for isolating nucleic acids from complex samples can comprise proteins or synthetic polymers that exhibit temperature triggered phase separation and that incorporate pH switchable ionizable groups. For example, the synthetic polymer can be a poly(N-isopropyl acrylamide) (PNIPAAm). An example of pH switchable ionizable groups that could be incorporated synthetically into PNIPAAm are imidazole groups such as the side chain of histidine.

In another example, the polymer can be an intrinsically disordered protein (IDP) comprising an amino acid composition that exhibits temperature triggered phase separation. Exemplary IDPs include polycationic ELPs, collagen, elastins, resilins, RRM-RGG and HCV Core proteins, and polypeptides comprising amino acid repeats rich in proline and glycine. The polypeptides can be modified or “tuned” to exhibit soluble to insoluble phase transitions that are of interest, including a lower critical solution temperature (LCST) transition that occurs upon heating above a critical solution temperature or an upper critical solution temperature (UCST) transition that occurs upon cooling below a critical temperature. See Quiroz, F., Chilkoti, A., Sequence heuristics to encode phase behavior in intrinsically disordered protein polymers, Nat. Mater. (2015); 14(11): 1164, which is incorporated herein by reference.

The phase behavior and NA binding affinity of a model polycationic ELP (called E3) is described herein. To produce E3, an otherwise uncharged ELP is engineered to contain equally spaced, interspersed cations that can promote electrostatic binding to nucleic acids after undergoing phase change. E3, with peptide sequence [(VPGXG)₁₀-GKG]₈, comprises 10 subunits of 8 concatenated neutral pentamers (VPGXG, X=8:2 ratio of Val/Ala), each flanked by cationic Lys residues. E3 undergoes simple coacervation, driven by a thermodynamic preference for homotypic self-interactions over heterotypic ones, in contrast to charge-mediated complex coacervation, in which oppositely charged polyanions associate to form coacervates in solution. Above a concentration dependent transition temperature (TT), the model cationic E3 protein undergoes LLPS in the presence or absence of DNA. Furthermore, the condensates formed by simple coacervation can be thermodynamically tuned with NaCl to preferentially interact with single stranded DNA to form synthetic deoxyribonucleoprotein (DNP) coacervates.

The DNA binding affinity of E3 and the amount of DNA captured and sequestered within E3 coacervates of distinct size and composition are measured systematically at different operating points by varying initial E3 concentration and the addition of charge shielding NaCl salt. An adapted mean field Flory-Huggins (FH) theory is used to mediate the strength of E3-DNA interaction by ionic strength through linearization of the Debye-Hückel free-energy in our evaluation of component FH interaction parameters. The FH interaction parameters are fit to fluorescence spectroscopy and microscopy data collected from bulk and from microdroplet samples to create ternary phase diagrams that interpret our experimental observations. Results showing dependence of FH interaction parameters with ionic strength are corroborated by the Debye-Hückel linearization. This combined approach results in the creation of phase diagrams that quantify DNA component partitioning within discrete protein- and solvent rich phases of known volume fraction across a range of salt and E3 compositions. The application of the method described herein provides a simple two-step DNA solution purification assay, with implications for applications such as viral RNA extraction, RNA/DNA capture from biological fluids, and gene regulation in synthetic cells.

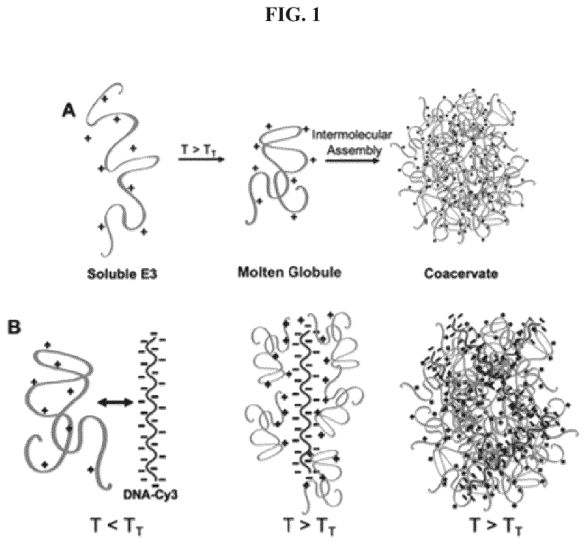

A schematic of E3 coacervate formation and capture of DNA is illustrated in FIG. 1. (A) Illustration of the engineered ELP called “E3” showing the distribution of positively charged Lys residues throughout the random coil protein polymer chain (left). When heated above the transition temperature (T>TT), the E3 chains collapse into molten globule species (middle) that coarsen to form E3-rich coacervates (right). (B) In the presence of negatively charged DNA species, reversible, electrostatically driven interactions between phase transitioned E3 and DNA can occur (left), where condensed E3 molecules bind with DNA (middle), and overtime capture DNA within fully coarsened coacervates (right).

TABLE 1
The amino acid sequence of elastin-like
polypeptide (ELP) “E3” is shown belowas SEQ ID
NO. 1.
3 Amino Acid Sequence
VPGVGVPGAGVPGVGVPGVGVPGVGVPGVGVPGAGVPGVGVPGVGVPGG
KGVGVPGVGVGAGVPGVGVPGVGVPGVGVPGVGVPGAGVPGVGVPGVGV
PGGKGVGVPGVGVPGAGVPGVGVPGVGVPGVGVPGVGVPGAGVPGVGVP
GVGVPGGKGVGVPGVGVPGAGVPGVGVPGVGVPGVGVPGVGVPGAGVPG
VGVPGVGVPGGKGVGVPGVGVPGAGVPGVGVPGVGVPGVGVPGVGVPGA
GVPGVGVPGVGVPGGKGVGVPGVGVPGAGVPGVGVPGVGVPGVGVPGVG
VPGAGVPGVGVPGVGVPGGKGVGVPGVGVPGAGVPGVGVPGVGVPGVGV
PGVGVPGAGVPGVGVPGVGVPGGKGVGVPGVGVPGAGVPGVGVPGVGVP
GVGVPGVGVPGAGVPGVGVPGVGVPGGKGY

The E3 polypeptide of SEQ ID NO. 1 can also be represented without the N-terminal MG or the C-terminal Y as SEQ ID NO. 2: [(VPGXG)₁₀-GKG]₈.

ssDNA Influences the Phase Behavior of Aqueous E3

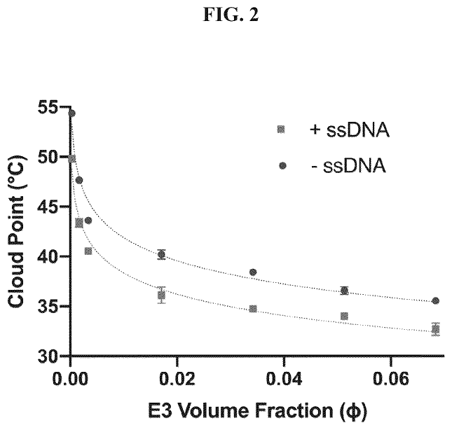

To quantify the effect of ssDNA (0.5 M, 28 nt) on the thermally dependent phase behavior of E3 (sequence: [(VPGXG)₁₀-GKG]₈ (SEQ ID NO: 2), X=8:2 ratio of Val:Ala), temperature-controlled spectrophotometry was used to measure cloud-point transition temperature (Tt) as a function of volume fraction (ϕ)—the fraction of solution volume occupied by E3 chains—for E3 in the presence or absence of ssDNA species (+ssDNA or −ssDNA, respectively). E3 Tt(ϕ) was measured for volume fractions ranging from 4=0.00034 to 4=0.068 in pH-stable buffer. The range of 4 values correspond to E3 concentrations of 0.01 mM to 2 mM. The E3 polymer maintains canonical ELP LCST dilute phase behavior—a decrease in Tt with increasing E3 volume ϕ—for both +ssDNA and −ssDNA solutions. For all replicate samples, it was found that the presence of ssDNA prompts a shift to lower Tt(ϕ) across all experimental 4 values when compared to Tt(ϕ) for the −ssDNA E3 replicate samples (FIG. 2). Average Tt with standard deviations are shown as a function of E3 volume fraction (ϕ) in the presence (squares) and absence (circles) of DNA (0.5 M, 28 nt) are plotted. The dashed lines represent the best logarithmic fit for ELP (E3) at + and −ssDNA conditions.

NaCl Mediates Recruitment of ssDNA into E3 Condensates

The temperature and salt mediated partitioning of ssDNA with phase transitioned E3 condensates were investigated. Microfluidic generated water-in-oil emulsions provide a useful view into the LLPS microenvironment as it relates to E3-ssDNA binding behavior. ELP phase transition was triggered within microdroplets by increasing the temperature (T=55° C.) above the Tt of 1 mM E3 (ϕ=0.034, Tt+ssDNA=35° C.) to generate coarsening spherical E3 condensates surrounded by solvent-rich regions. Observing the evolution of the thermally phase transitioned system from 0 to 15 minutes, the coarsening of unlabeled E3 was tracked to completion and the concurrent partitioning of fluorophore labeled ssDNA-Cy3 in each phase by brightfield and fluorescence microscopy, respectively. The interaction between droplet encapsulated components of 1 mM E3 (brightfield), 0.5 μM ssDNA-Cy3 (fluorescence), and the influence of 0 or 100 mM NaCl salt (−NaCl and +NaCl, respectively) in aqueous droplets is detailed. For both − and +NaCl samples, representative droplet microscopy images are shown at: (1) t=0 minutes at room temperature, T<Tt, where E3 is in a soluble state; (2) t=3 minutes of incubation at T>Tt during the early stages of phase separation and E3 condensate coarsening; and (3) t=15 minutes at T>Tt when full coarsening and complete phase separation is achieved.

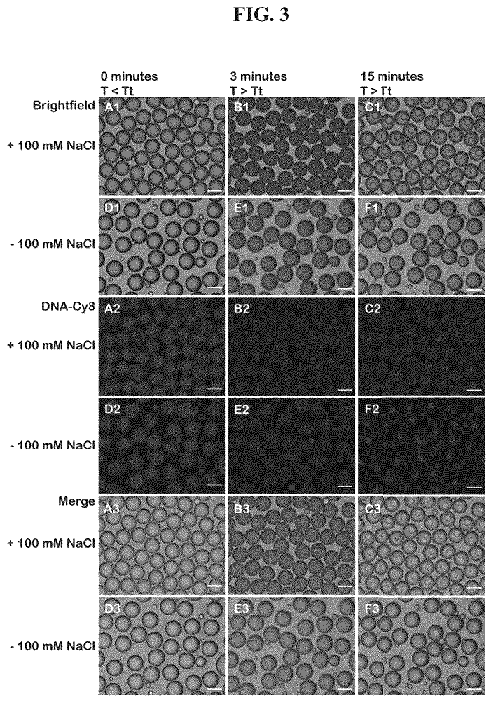

Across all t=0 samples at room temperature (T<Tt) no E3 condensates are observed (FIG. 3, A1 and D1) and all solution components are miscible and evenly distributed throughout the droplet volume (FIG. 3, A2, D2, A3 and D3). The incipient stages of thermally triggered E3 phase transition occur simultaneously throughout droplet volumes, corroborating demixing through spinodal decomposition for both +NaCl and −NaCl samples (FIG. 3, B1 and E1). After coarsening completes, each microdroplet exhibits a monodisperse core-shell arrangement in which the spherical core is an E3-rich condensate, and the surrounding shell is an aqueous rich phase (FIG. 3, C1 and F1). Time lapse fluorescence microscopy of ssDNA-Cy3 reveals that above Tt and without added NaCl, the fluorescent ssDNA-Cy3 is efficiently captured and localized within E3 condensates-noticeable even during the early stages of spinodal decomposition (FIG. 3, E2 and E3). Note that the recruitment of ssDNA-Cy3 into the E3 rich condensates is E3 concentration dependent. By contrast, addition of NaCl results in electrostatic shielding, partitioning more ssDNA-Cy3 in the solvent-rich phase (FIG. 3, B2 and C2). The combined E3 phase behavior and ssDNA-Cy3 partitioning for −NaCl and +NaCl is further evidenced by inspection of the merged brightfield and fluorescence microscopy images of FIG. 3, panels A3-F3.

### Flory-Huggins Phase Diagrams Describe NaCl Mediated DNA Capture by E3 Condensates

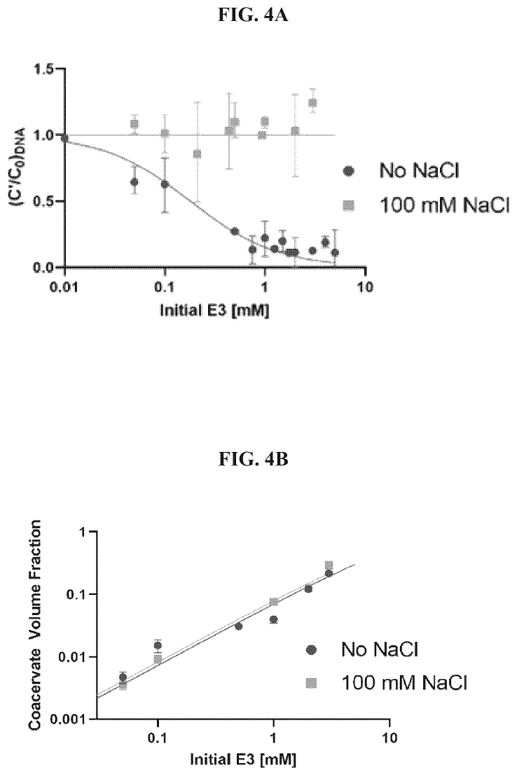

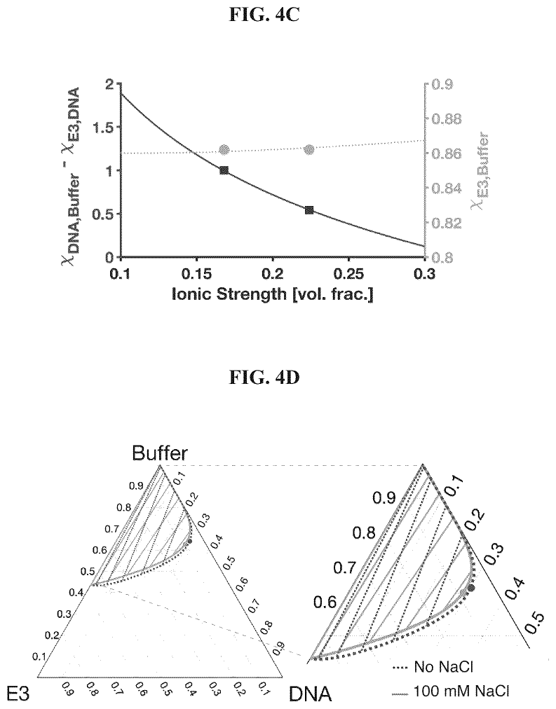

Using FH formalisms (see equations 1-4), the phase diagrams of solution E3 and DNA were approximated at two salt concentrations of 0 mM NaCl and 100 mM NaCl. To quantify the partitioning of DNA into E3 coacervates at 0 mM and 100 mM NaCl, fluorimetry was used to determine DNA concentration in the dilute solvent-rich phase (FIG. 4A) and microscopy was used to determine the total volume fraction occupied by E3 coacervates as measured in microdroplets (FIG. 4B), for a range of E3 concentrations (T=55° C.). Hence, the experimentally available data for fitting the FH parameters

χ={χ_{E3,Buffer},χ_{DNA,Buffer},χ_{E3,DNA}}

are the concentration variant supernatant DNA concentration ratio from initial

$${( \frac{{C}^{′}}{{C}_{0}} )}_{D N A}$$

and the total volume fraction of the E3 coacervate

$$p = \frac{{ϕ}_{E 3} - {ϕ}_{E 3}^{′}}{{ϕ}_{E 3}^{″} - {ϕ}_{E 3}^{′}} = \frac{{ϕ}_{D N A} - {ϕ}_{D N A}^{′}}{{ϕ}_{D N A}^{″} - {ϕ}_{D N A}^{′}} .$$

In FH theory, component i volume fraction is related to concentration by the molar volume of the buffer (v) by

$${ϕ}_{i} = \frac{v {N}_{i} {C}_{i}}{1 + v {\sum }_{i} {N}_{i} {C}_{i}} .$$

Without independent phase data of DNA in buffer, the DNA-buffer interaction parameter χ_{DNA,Buffer} is unknown. Keeping the difference χ_{DNA,Buffer}−χ_{E3,DNA} constant while varying χ_{E3,DNA} appears to generate nearly equivalent dilute phase DNA volume fractions. Therefore, keeping χ_{DNA,Buffer}=0 while finding the best fitting χ_{E3,DNA} will generate the best set of χ knowing that only χ_{DNA,Buffer}−χ_{E3,DNA} is expected to be unique. The solution E3 interaction parameter at T=55° C. was reported in our previous work χ_{E3,Buffer}=0.862 and is assumed to be constant for both salt conditions. For approximation, the degree of polymerization of the E3 was assumed to be equal to the number of pentameric repeats of the protein (i.e., VPGXG), in this case N_{E3}=80. For the DNA degree of polymerization, that molar volume ratio is assumed to be equal to molecular weight ratio between molecules, giving

$${N}_{D N A} = \frac{{M}_{D N A}}{{M}_{E 3}} {N}_{E 3} \approx 20 .$$

Fitting mean field Flory-Huggins (FH) theory equations (Equations 1-4 of Example 1E below) to the dilute phase DNA concentration ratio data simultaneously with the coacervate volume fraction data enables determination of the cross-polymer interaction parameter χ_{E3,DNA} and the buffer molar volume v at 0 mM NaCl and 100 mM NaCl conditions. Specifically, phase diagram tie line interpolation was used to find best fitting buffer molar volume for a variance of χ_{E3,DNA} for both measurements. Then, the intersection of the best fitting lines from each measurement determined the overall best fit. It was determined that 0 mM NaCl results in the interaction parameter X_{E3,DNA}=−1.0 and the 100 mM NaCl resulted in a larger χ_{E3,DNA}=−0.54. The interpolation of the tie lines from the resulting phase diagrams of the experimental concentrations used are presented in FIG. 4A showing the depression of DNA in the supernatant phase for the 0 mM NaCl condition, corresponding to enhanced DNA partitioning in the E3 coacervate, and the constant ratio of DNA for the 100 mM NaCl condition. These results agree with observations described in FIG. 3.

The best fitting buffer molar volume v was found to be 0.54 M⁽⁻¹⁾ and 0.58 M⁽⁻¹⁾ for the − and +NaCl respectively, and the mean value of 0.56 M⁽⁻¹⁾ was used for analysis. With respect to the experimental variance of the microdroplet measurements of FIG. 4B, a small change from addition of 100 mM NaCl suggests a nominal effect of excluded volume from increasing ionic concentration of a buffer solution, which is corroborated from literature. Furthermore, the value for buffer molar volume should be recognized as being referenced by the assumed chain length. As the chain length of E3 was assumed to be the number of pentameric repeats, N_{E3}=80, this molar volume suggests that each VPGXG unit will occupy 560 cm³/mol or 112 cm³/mol for each amino acid.

An appendage to FH could be used to describe the change in interaction parameters leading to partitioning of DNA with E3 coacervates at various NaCl concentrations. It is assumed that the ionic effects are captured on a mean field level by introducing an enthalpic free energy provided by the Debye-Hückel (DH) theory. Considering the solvation criteria for DH, it should be admissible to assume the ionic effects contribute by appending the standard FH interaction parameters with a linear perturbation for charge effects. Each effective interaction parameter χ_{i,j} will be, by a first approximation, the hypothetical interaction parameter of the same system without Coulombic interactions χ_{i,j}⁰ appended with the respective term from a linearization of the DH free energy.

$${χ}_{E 3 , Buffer} = {χ}_{E 3 , Buffer}^{0} + 2 A \frac{\sqrt{{I}_{s}}}{1 + \sqrt{{I}_{s}}} ( {I}_{s} - {α}_{E 3} ) \qquad ( 5 )$$

$${χ}_{DNA , Buffer} = {χ}_{DNA , Buffer}^{0} + 2 A \frac{\sqrt{{I}_{s}}}{1 + \sqrt{{I}_{s}}} ( {I}_{s} - {α}_{DNA} ) \qquad ( 6 )$$

$${χ}_{E 3 , DNA} = {χ}_{E 3 , DNA}^{0} - ( \frac{( {α}_{E 3} - {I}_{s} ) ( {α}_{DNA} - {I}_{s} )}{\sqrt{{I}_{s}} {( 1 + \sqrt{{I}_{s}} )}^{2}} + \frac{2 \sqrt{{I}_{s}}}{1 + \sqrt{{I}_{s}}} ( 2 {I}_{s} - {α}_{E 3} - {α}_{DNA} ) ) \qquad ( 7 )$$

Here, A is the Debye-Hückel free-energy pre-factor which is related to ionic radius. Iₛ is the ionic strength of the salt solution multiplied with the buffer molar volume v. The Iₛ value for our 100 mM di-basic sodium phosphate buffer at 0 and 100 mM added NaCl is 0.3M and 0.4M, respectively. The component of ionic strength for each polyion is given as

$${α}_{i} = \frac{{z}_{i}^{2}}{2 {N}_{i}} ,$$

where zᵢ is the total number of charges of molecule i.

FIG. 4C shows the dependence on NaCl of DH modified FH interaction parameters and agrees with the experimentally determined interaction parameters. The other fitted parameters are the DH free energy pre-factor A=0.1 and the non Columbic interaction parameters χ_{E3,Bufffer}⁰=0.874 and χ_{DNA,Bufffer}⁰−χ_{E3,DNA}⁰=2.71. The small variance for χ_{E3,Buffer} is the likely result of a small ionic strength component of E3 α_{E3}=0.4 as the change from − to +NaCl leads to a total change Δχ_{E3,Buffer}=0.002. This contrasts with the larger change from the DNA—Buffer interaction parameter with α_{DNA}=19.6 and Δχ_{DNA,Buffer}=−0.12. However, these differences are expected to be strongly dependent on the choice for polymer chain length, as this value will directly affect the resulting buffer molar volume.

Fully determining the FH parameters N and χ allows for the determination of the full three component phase diagram, including the binodal, tie lines and critical point, and these plots are given in FIG. 4D. It should be noted that the underlying interaction parameters were determined in a dilute DNA limit and the extrapolation to higher concentration has not been validated. Nonetheless, these plots show the fundamental contrast that increasing salt concentration has on DNA phase partitioning with E3. Namely, increasing salt concentration will lower the equilibrium concentration of DNA in the E3 coacervate. This behavior is illustrated by dissimilar tie line slopes for − and +NaCl, where intersections with the binodal gives the equilibrium component volume fraction in each phase. Furthermore, this observation is corroborated by Debye-Hückel theory.

Sequential LLPS Allows Recovery of DNA from E3/DNA Solutions

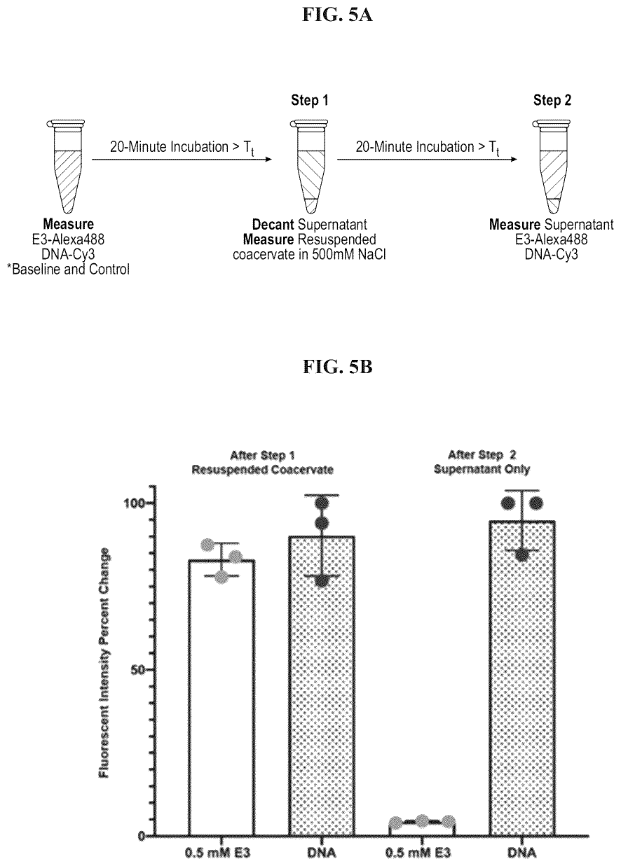

The fact that E3 efficiently captures DNA upon coacervation in the absence of added salt suggests a potential simple method for separation of nucleic acids from solutions by LLPS. As depicted in FIG. 5, a two-step/two-color assay was designed and validated to quantify the concentration of DNA isolated from a starting mixed sample of E3 and DNA using fluorimetry. Briefly, the fluorescence of 0.5 mM E3 doped with E3-labeled was measured by Alexa 488 (E3-488) along with 500 nM DNA-Cy3 at room temperature (FIG. 5A). Next, the solutions were incubated at 55° C. to induce E3/DNA phase separation. As observed in FIG. 3, the DNA localizes to the protein-rich (coacervate) phase upon this first LLPS process. The supernatant was then discarded and the coacervate resuspended with 500 mM NaCl (Step 1 in FIG. 5) and fluorescence intensities were measured (FIG. 5B, left bars); over 80% of E3 and DNA are retained through this process. Next, in Step 2, the resuspended coacervate was incubated at 55° C. to induce phase transition of E3 in the presence of 500 mM NaCl. After this second round of LLPS, the fluorescence of the supernatant was measured and the DNA-Cy3 intensity was found to be comparable to the original DNA-Cy3 measured before the first incubation. Meanwhile, the signal of E3-488 decreases by 95% under these conditions, demonstrating an efficient separation and recovery of DNA from protein solution upon the addition of salt (FIG. 5B, right bars). These results agree with findings detailed in FIG. 3 and FIG. 4. FIG. 4A demonstrates that initial (i.e., prior to LLPS) ELP concentration should be equal to, or in excess of, 0.5 mM for efficient capture of 500 nM DNA within the coacervate phase under low salt conditions. These results suggest that triggered LLPS of NA-binding IDPs is an efficient method of isolating nucleic acids from complex solutions that avoid the use of expensive consumables such as spin columns or magnetic beads.

In sum, the phase behavior and molecular partitioning of a ternary component ELP, DNA, and aqueous buffer solution system was characterized. A model ELP called E3 was engineered that comprises ELP blocks flanked by 8 evenly spaced, cationic lysine residues (E3 sequence of SEQ. ID. NO. 2: [(VPGXG)₁₀-GKG]₈ (SEQ ID NO: 2), X=8:2 ratio of Val:Ala). The concentration dependent lower critical solution temperature (LCST) transition temperature of E3 is reduced by a few degrees Celsius in the presence of DNA. The NaCl-mediated capture of fluorescently labeled Cy3 DNA by E3 condensates is observed in microfluidic generated drops and characterized by fluorescence spectroscopy. Results above show E3 efficiently captures DNA upon coacervation only in the absence of added NaCl salt and at 100 mM added NaCl, DNA shows no preference for the coacervate or solvent-rich phase. Mean field Flory-Huggins (FH) theory describes the drastic reduction in DNA partitioning by E3 coacervates with addition of 100 mM NaCl.

Multivariate fitting of FH interaction parameters was applied to experimental data of concentration variant supernatant DNA concentration ratio from the initial and the total volume fraction of E3 coacervates. A linearized Debye Hückel term was introduced to FH interaction parameters to account for variable E3 condensate DNA capture as a result of change in ionic strength. Results above show similar changes in the DH-modified FH interaction parameters as those estimated from fitting to experimental data. Ternary phase diagrams complete with tie lines and binodal curves were generated that quantify DNA and E3 component partitioning within protein- and solvent-rich phases at 0 mM and 100 mM added NaCl buffer conditions. Finally, the utility of our system was demonstrated by prototyping a new DNA purification assay by using thermal LLPS of E3 and addition of NaCl salt to control the DNA binding and release behavior of E3 condensates.

### Nucleic Acid-Containing Samples

As described herein, the isolation of nucleic acids using IDP coacervates may be applied to a variety of nucleic acid-containing samples. For example, the sample can be a physiologically relevant sample such as body tissue or body fluids. The body fluids can include saliva, sputum, mucus, nasopharyngeal discharge (e.g., nasal discharge collected from a patient by nasopharyngeal swab), blood, serum, plasma, urine, aspirate, stool or a combination thereof. The sample can also be from environmental sampling such as municipal wastewater, swabs from contaminated surfaces, and air samples (e.g., SKC polytetrafluoroethylene (PTFE) filter cassette samples).

### Nucleic Acid-Based Diagnostics

The nucleic acids isolated in the coacervate produced by the IDPs described herein can be subjected to a variety of nucleic acid-based diagnostic assays. Such diagnostic assays can be implemented to identify diseases such a pathogenic bacteria or viruses in the coacervate. For example, many nucleic acid-based diagnostics rely on the quantitative polymerase chain reaction (qPCR) or real-time quantitative reverse transcription PCR, which have been widely adopted and are frequently used in clinical laboratories. The versatility, robustness and sensitivity of PCR have made this technology commonly used for the detection of DNA and RNA biomarkers.

In non-PCR based methods, nucleic acid-based diagnostics can include a variety of methods for amplifying nucleic acids including isothermal amplification, nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR), and strand displacement amplification (SDA) based diagnostics. See, for example, Kaminski, M. et. al., CRISPR-based diagnostics, Nature Biomedical Engineering (2021) 5: 643-656.

### Kits

The present technology further pertains to a packaged pharmaceutical composition such as a kit or other container for detecting, controlling, preventing, or treating a disease. The kits of the technology can be designed for detecting, controlling, preventing, or treating diseases such as those described herein (e.g., a viral infection). In one embodiment, the kit or container can hold the intrinsically disordered protein (IDP), such as the polycationic elastin-like polypeptide, as well as instructions for preparing a composition that includes the polycationic elastin-like polypeptide.

The kits of the technology can also comprise containers with tools useful for administering the compositions of the technology. Such tools can include syringes, swabs, catheters, antiseptic solutions, and the like. Some kits can include all of the desired tools, solutions, compounds, including mixing vessels, utensils, and injection devices, to diagnose or treat a patient according to any of the methods described herein. In one embodiment, a kit includes the IDP of the various embodiments described herein. The IDP can be sterile-packaged as a dry powder in a suitable container (e.g., a substantially water-impermeable) such as a syringe, vial (e.g., the vial can include a septum and/or a crimp seal; and the vial can optionally comprise an inert atmosphere, such as a nitrogen atmosphere or dry air) or pouch (e.g., a pouch comprising a moisture barrier; and the pouch can optionally comprise an inert atmosphere, such as a nitrogen atmosphere, or dry air). The kit can also include a desiccant. The desiccant can be included in the pouch or integrated into the layers of the pouch material. In some embodiments, the IDP can be sterile-packaged in frozen vehicle. As mentioned previously, the vehicle can be any suitable vehicle, including flowable vehicles (e.g., a liquid vehicle) such as a flowable, bioresorbable polymer, saline, sterile water, Ringer's solutions, and isotonic sodium chloride solutions. Examples of vehicles include, but are not limited, to Sodium Chloride Injection USP (0.9%), Ringer's Injection USP, Lactated Ringer's Injection USP, Sodium Lactate Injection USP, Dextrose Injection USP (5% or 10%), Bacteriostatic Water for Injection USP and Sterile Water for Injection USP. In some examples, the IDP can be suspended in a buffer; pre-filled into a container, such as a syringe; and frozen.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to formula (I) are equally envisaged as being applicable to formula (1b).

The technology will be further described by the following non-limiting examples.

### EXAMPLES

### Example 1: Methods

A. Expression and Purification of E3.

The expression vector pET24 was purchased from Novagen, Inc. (Milwaukee, WI). One-Shot BL21 Star (DE3) Escherichia coli cells were from ThermoFisher Scientific (Waltham, MA). Restriction enzymes were from New England Biolabs (Beverly, MA). DNA purification kits were purchased from QIAGEN, Inc. (Valencia, CA). DNA sequences (genes fragments and ssDNA) were purchased from Integrated DNA Technologies (Coralville, IA). tRNA was purchased from Millipore Sigma (St. Louis, MO). Luria broth (LB) agar plates were purchased from Bacto Agar, Becton Dickinson (Franklin Lakes, NJ), and Millipore Sigma (St. Louis, MO). Kanamycin was from Ultrapure, VWR, (Radnor, PA). LB Broth and Terrific Broth (TB) was from IBI Scientific (Dubuque, Iowa). The viral RNA isolation kit was from Zymo Research (Irvine, CA). Reagents for RT-qPCR were obtained from ThermoFisher Scientific (Waltham, MA).

The gene encoding the E3.10 protein was constructed using plasmid pET24-E3 as a starting point. The RRM and RGG domains of the FUS proteins are engineered into the E3 protein using the Golden Gate assembly method as described. Engler, C.; Kandzia, R.; Marillonnet, S. A One Pot, One Step, Precision Cloning Method with High Throughput Capability. PLoS ONE 2008, 3 (11), e3647. doi.org/10.1371/journal.pone.0003647. Briefly, the E3 plasmid and the FUS protein plasmid was digested with BsaI, and subsequently ligated together to generate pET24-E3.10. pET24-E1.40COR30 was constructed by ligating a synthetic COR30 sequence (Integrated DNA Technologies, Coralville, IA) to the 3′ end of the E1.40 sequence in pET24-E1.40 using a single step recursive ligation method. McDaniel, J. R.; MacKay, J. A.; Quiroz, F. G.; Chilkoti, A. Recursive Directional Ligation by Plasmid Reconstruction Allows Rapid and Seamless Cloning of Oligomeric Genes. Biomacromolecules 2010, 11 (4), 944-952. doi.org/10.1021/bm901387t. Plasmids expressing H-20 and H-24 were constructed following previously described methods. MacKay, J. A.; Callahan, D. J.; FitzGerald, K. N.; Chilkoti, A. Quantitative Model of the Phase Behavior of Recombinant PH-Responsive Elastin-Like Polypeptides. Biomacromolecules 2010, 11 (11), 2873-2879. https://doi.org/10.1021/bmi100571j.

Escherichia coli (BL21) cells harboring plasmids encoding the protein of interest were inoculated onto LB agar plate containing 45 μg/mL kanamycin sulfate and incubated overnight at 37° C. Starter cultures grown from individual colonies were used to inoculate 3 mL of LB broth with 45 μg/mL kanamycin sulfate. This culture was incubated overnight at 220 rpm and 37° C. The culture was then transferred into 1 L of TB supplemented with 45 μg/mL kanamycin sulfate. Cultures were incubated at 37° C. with agitation for 6 hrs before induction with isopropyl β-d-1-thiogalactopyranoside (IPTG). The culture was induced at 37° C. for 18 hrs prior to harvest by centrifugation at 4° C. and 3000 rpm for 30 min. The resulting pellets were resuspended into a lysis buffer (phosphate buffered saline (1×PBS), 1 Pierce™ protease inhibitor tablet from ThermoFisher (Waltham, MA), and 0.05 mM, ethylenediamine tetraacetic acid (EDTA) at pH 8.0) and lysed by sonication to release all intracellular content.

Expressed proteins were purified by inverse transition cycling, exploiting the reversible thermally responsive protein phase separation of the ELP constructs. This approach comprises cyclic centrifugation steps that alternate between cold (4° C.) and hot (40° C.) centrifugation in PBS until all contaminants are removed, usually within 2-5 cycles. For E3.10, hot centrifugation was replaced by room temperature centrifugation. LLPS was triggered by the addition of 1M ammonium sulfate to cool the solution from 37° C. to 25° C. to induce ELP phase separation and to avoid possible denaturation of folded domains in the protein.

B. Measurement of E3 Cloud Point TT in the Presence of DNA by UV-vis Spectroscopy. Compositions of DNA species used in the experiments described provided in Table 2 below.

TABLE 2
Sequence of DNA used in experiments described herein.

| DNA | | | |
|---|---|---|---|
| Name | Type | Sequence | Length |
| Cy3- | Single | Cy3-5′- | 28 nt |
| SSDNA | Stranded | TTTTTCCTAGAGAGTAGAGCCTGCTTCG-3′ | |
| “DNA- | DNA | (SEQ ID NO: 12) | |
| Cy3” | | | |
| SSDNA | Single | 5′-TTTTTCCTAGAGAGTAGAGCCTGCTTCG- | 28 nt |
| | Stranded | 3′ (SEQ ID NO: 13) | |
| | DNA | | |

Samples containing a range of E3 concentrations, 500 nM of single stranded 28 nucleotide (nt) DNA (Integrated DNA Technology, Coralville, Iowa), and buffer were prepared according to Table 3. The samples for temperature dependent absorbance experiments are described in Table 3, with the E3 content given by volume fraction and concentration. All samples were prepared in 100 mM NaH2PO4.

TABLE 3
Samples analyzed by temperature-programmed spectrophotometry.

| Sample | E3 Volume Fraction (ϕ) | [E3] mM | [ssDNA] μM |
|---|---|---|---|
| 1* | 0.017 | 0.5 | 0 |
| 2** | 0 | 0 | 0.5 |
| 3 | 0.00034 | 0.01 | 0.5 |
| 4 | 0.0017 | 0.05 | 0.5 |
| 5 | 0.0034 | 0.1 | 0.5 |
| 6 | 0.017 | 0.5 | 0.5 |
| 7 | 0.034 | 1 | 0.5 |
| 8 | 0.051 | 1.5 | 0.5 |
| 9 | 0.068 | 2 | 0.5 |
| 10 | 0.00034 | 0.01 | 0 |
| 11 | 0.0017 | 0.05 | 0 |
| 12 | 0.0034 | 0.1 | 0 |
| 13 | 0.017 | 0.5 | 0 |
| 14 | 0.034 | 1 | 0 |
| 15 | 0.051 | 1.5 | 0 |
| 16 | 0.068 | 2 | 0 |

*Positive control;
**Negative control

The transition temperature was quantified by measuring the absorbance of the samples at 380 nm, without and with 500 nM ssDNA, as a function of temperature with a temperature controlled (Peltier temperature controller, Agilent, Santa Clara, CA) UV-vis spectrophotometer (Cary 300 UV-vis, Agilent). The data are then plotted to display the change in absorbance of the solution over a temperature range of 30-60° C. and the TT is obtained by taking the maximum in the first derivative of the absorbance as a function of temperature.

C. Characterization of DNA Concentration by Fluorescence Spectroscopy.

A temperature and time dependent fluorescence spectroscopy assay was used to characterize the binding interactions between E3 and DNA-Cy3 (28 nt single stranded oligonucleotide with cyanine-3 fluorophore attached to the 5′ end, shown in Table 2) in the presence or absence of 100 mM NaCl (VWR). 1 mL of total volume sample solutions in triplicate, at either 0 or 100 mM NaCl, were prepared. The experimental samples contain E3 at varying concentrations, 500 nM DNA-Cy3, 100 mM sodium phosphate buffer (sodium phosphate powder, Sigma Aldrich, St Louis, Missouri), and molecular biology-grade water (Corning) at pH 7.0 to maintain E3 phase transition behavior, charge of the Lys residues distributed within the E3 polymers in solution, and a stable pH. All samples were prepared using dark, LightSafe 1.5 mL polypropylene microcentrifuge tubes (Sigma-Aldrich). Control samples were prepared and treated as experimental samples to control stability of the fluorescence intensity of Cy3 and used to normalize the measured fluorescence intensity values. The solutions were vortexed and centrifuged for 5 seconds to combine, then pipetted at room temperature (23° C.) into 100 μL precision volume quartz cuvettes (Ultra-Micro Cell 105.250-QS LP 10 mm×2 mm, CH 8.5 mm, Hellma Analytics, Plainview, NY). The fluorescence intensity of the samples was measured using a fluorimeter (PTI QuantaMaster QM-400 Horiba, Irvine, CA) with 520 nm excitation wavelength and 540-650 nm emission scan settings. Next, the sample volumes were transferred from the cuvettes back into the dark microcentrifuge tubes to be incubated at 55° C. in a heating block (Isoblock Dry Bath Heat Block, Benchmark, Edison, NJ) for 2 h to induce phase separation of E3 that results in the formation of a clear protein-poor phase and a protein-rich phase settled into the bottom of the tube. By deliberate pipetting, the supernatant-only (protein-poor phase) was transferred to the quartz cuvettes, leaving the coacervate phase undisturbed at the bottom of the tube. The fluorescence intensity of the supernatant was measured by fluorimetry with the same aforementioned settings. This enables the determination of the amount of DNA-Cy3 present in the supernatant versus the amount of DNA-Cy3 associated with E3 in the coacervate phase as plotted in FIG. 4A. The same procedure is applied to track the location of E3 and DNA in the DNA purification assay depicted in FIG. 5A.

A two-step/two-color isolation assay was designed and validated to quantify the concentration of DNA isolated from a starting mixed sample of E3 and DNA using fluorimetry. Briefly, the fluorescence of E3 doped with E3-Alexa488 was measured, with a 450 nm excitation wavelength and 470-540 nm emission scan settings, along with 500 nM DNA-Cy3 at room temperature (FIG. 5A). Next, the solutions were incubated at 55° C. to induce E3 phase separation. Two phases form and the supernatant of the solution was decanted and the coacervate resuspended with 500 mM NaCl to measure by fluorimetry to track the location of the green emission from E3-488 versus the red emission from DNA-Cy3.

D. Fluorescence Microscopy Imaging.

To image the process of temperature-mediated E3 coacervation and interaction with DNA-Cy3, an Olympus IX83 fluorescence microscope (Olympus Life Science Technology Division, Center Valley, PA) was equipped with a Physitemp cooling and heating stage (TS4-MP/ER/PTU, Clifton, NJ) fitted with a temperature controller. Microfluidic droplets were generated using previously described methods and droplet generator. These droplets contain different E3 concentrations, 500 nM DNA-Cy3, sodium phosphate buffer at pH 7.0, and either 0 or 100 nM added NaCl, and are pipetted onto a glass slide (18 mm×18 mm Square Micro Cover Glass, VWR). The droplet population was allowed to settle for 5 min until there is a single layer of droplets on the surface. The glass slide was mounted onto the temperature-controlled stage and equilibrated to 20° C. Images were acquired by a high dynamic range camera (ORCA-Flash4.0 V3 Digital CMOS camera C13440-20CU, Hamamatsu, Bridgewater, NJ) using both brightfield and fluorescence (520 nm LED excitation source/550 nm emission filter) acquisition modes, across a temperature range including values below (25° C.), and above the TT (55° C.) (FIG. 3) until complete LLPS is achieved.

E. Ternary Component Flory-Huggins Phase Diagrams.

Using mean field Flory-Huggins (FH) theory, ternary phase diagrams can be created to quantify a DNA component partitioning within discrete protein and solvent rich phases across a range of salt and E3 compositions. The standard FH equation providing the Helmholtz free energy density f for an incompressible two-polymer aqueous system of polymer volume fraction components ϕ₁ and ϕ₂ is

$$f = \frac{{ϕ}_{1} ln {ϕ}_{1}}{{N}_{1}} + \frac{{ϕ}_{2} ln {ϕ}_{2}}{{N}_{2}} + {ϕ}_{s} ln {ϕ}_{s} + {χ}_{1} {ϕ}_{1} {ϕ}_{s} + {χ}_{2} {ϕ}_{2} {ϕ}_{s} + {χ}_{x} {ϕ}_{1} {ϕ}_{2} \qquad ( 1 )$$

where χᵢ are pairwise interaction parameters with the solvent and χₓ is the interaction parameter for components 1 and 2. The degree of polymerization N₁ and N₂ are the ratios, with respect to solvent, of the molar volumes of E3 and DNA, respectively. The solvent volume fraction ϕ₅ is given by the volume fraction conserving condition 1=Σᵢϕᵢ. The criteria for phase equilibria in a multicomponent system is the equality of chemical potential (given as

$${μ}_{i} = \frac{\partial f}{\partial {ϕ}_{i}} )$$

between all phases for each component. With the volume fraction conservation constraint, the buffer chemical potential is no longer independent of the other components. In its place analytically is the constraint of equivalent excess grand free-energy between phases, which is otherwise known as a Weierstrass-Erdmann condition. These criteria are summarized as

μ₁(ϕ₁′,ϕ₂′)=μ₁(ϕ₁″,ϕ₂″)=μ₁′ (2)

μ₂(ϕ₁′,ϕ₂′)=μ₂(ϕ₁″,ϕ₂″)=μ₂* (3)

f(ϕ₁′,ϕ₂′)−f(ϕ₁″,ϕ₂″)=(ϕ₁′−ϕ₁″)μ₁*+(ϕ₂′−ϕ₂″)μ₂* (4)

Here ϕᵢ′ and ϕᵢ″ denote the volume fraction of component i in the dilute and dense phases, respectively. There exists one set of the binodal chemical potentials {μ₁*, μ₂*} for each tie line within the phase envelope, given explicitly as the set {ϕ₁′, ϕ₁″, ϕ₂′, ϕ₂″ }. Therefore, the coupled set of equations can be determined experimentally by determining {ϕ₁′, ϕ₁″, ϕ₂′, ϕ₂″ } for a unique set of FH parameters.

### Example 2: Sequence of RNA-Binding IDPs

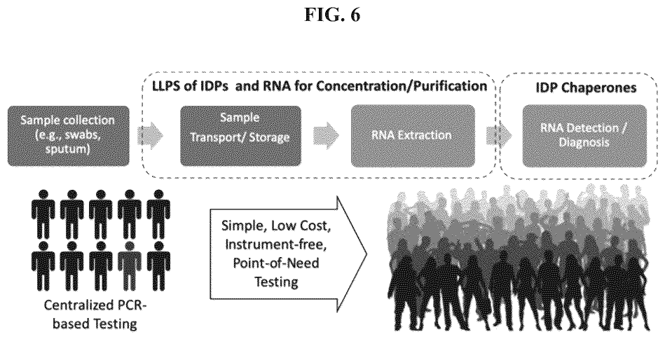

Intrinsically disordered proteins (IDPs) used to target viral RNA can circumvent inherent drawbacks of existing methodologies for highly efficient and rapid isolation of viral RNA from complex samples. Engineered IDPs can isolate viral RNA by phase separation in complex samples for viral RNA for detection and diagnosis, as illustrated in FIG. 6:

In some cases the IDP can be an ELP having an amino acid sequence of SEQ ID NO.: 3: (Val-Pro-Gly-X-Gly)ₙ, wherein X is any amino acid residue except Proline. The IDP can have a thermally reversible lower critical solution temperature (LCST) phase transition at temperature (Tt). The thermally responsive properties of the ELP are influenced by the number of ELP pentapeptides and the identity of the amino acid X in SEQ ID NO. 2.

In some cases, the IDP can be an ELP that includes the amino acid sequence of SEQ ID NO. 1: [(VPGXG)₁₀-GKG]₈ or SEQ ID NO. 2, as shown in Table 1 above. FIG. 7 illustrates an IDP comprising SEQ ID NO. 6 below (shown as “E3”) followed by an 87 amino acid RNA recognition motif (“RRM”) from Fused in Sarcoma (FUS) protein which comprises an RNA binding folded domain. The RNA recognition motif can be followed by an RNA binding disordered region comprising a 51 amino acid Arginine/Glycine rich domain from the FUS protein (“RGG”).

In some cases, the IDP can be a 124 amino acid full-length hepatitis C virus core protein (COR124) (nucleocapsid) of SEQ. ID. NO. 11:

MSTNPKPQRKTKRNTNRRPQDVKFPGGGQIVGGVYLLPRRGPRLGVRAT
RKTSERSQPRGRRQPIPKARRPEGRTWAQPGYPWPLYGNEGCGWAGWLL
SPRGSRPSWGPTDPRRRSRNLGKVID

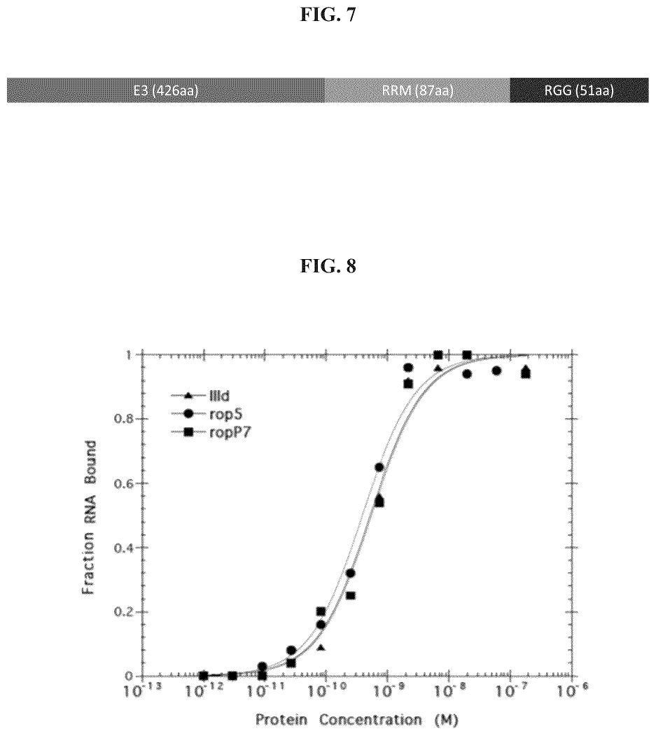

This sequence comprises a partially disordered, highly charged and robust nucleic-acid binding protein. The RNA binding profile of COR 124 as a function of concentration is shown in FIG. 8.

In some cases, the IDP can be an ELP having an amino acid sequence of SEQ ID NO. 4: (VPGVG)₄₀. The ELP of SEQ ID NO. 4 can be followed by a 30 amino acid sequence from HCV Core protein (COR 30) that has no cystine residues and binds RNA. For example, an ELP followed by COR30 with a length of 231 amino acids and a molecular weight of 19.9 kDA has the amino acid sequence of SEQ ID NO. 5:

MSKGPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGV
GVPGVGVPGVGVPGVGVPGVGVPGVGVPGVGVPGVGVPGVGVPGVGVPG
VGVPGSTNPKPQRKTKRNTNRRPQDVKFPGGGQIV

In some cases, the IDP can be an ELP having an amino acid sequence of SEQ ID NO. 1, followed by the RRM and the RGG. The resulting ELP has SEQ ID NO. 6:

MGVGVPGVGVPGAGVPGVGVPGVGVPGVGVPGVGVPGAGVPGVGVPGVG
VPGGKGVGVPGVGVGAGVPGVGVPGVGVPGVGVPGVGVPGAGVPGVGVP
GVGVPGGKGVGVPGVGVPGAGVPGVGVPGVGVPGVGVPGVGVPGAGVPG
VGVPGVGVPGGKGVGVPGVGVPGAGVPGVGVPGVGVPGVGVPGVGVPGA
GVPGVGVPGVGVPGGKGVGVPGVGVPGAGVPGVGVPGVGVPGVGVPGVG
VPGAGVPGVGVPGVGVPGGKGVGVPGVGVPGAGVPGVGVPGVGVPGVGV
PGVGVPGAGVPGVGVPGVGVPGGKGVGVPGVGVPGAGVPGVGVPGVGVP
GVGVPGVGVPGAGVPGVGVPGVGVPGGKGVGVPGVGVPGAGVPGVGVPG
VGVPGVGVPGVGVPGAGVPGVGVPGVGVPGGKGYNTIFVQGLGENVTIE
SVADYFKQIGIIKTNKKTGQPMINLYTDRETGKLKGEATVSFDDPPSAK
AAIDWFDGKEFSGNPIKVSFATRRADFNRGGGNGRGGRGRGGPMGRGGY
GGGGSGGGGRGGFPSGGGGGGGQQR

### Example 3: Nucleic Acid Isolation Using Liquid-Liquid Phase Separation (LLPS)

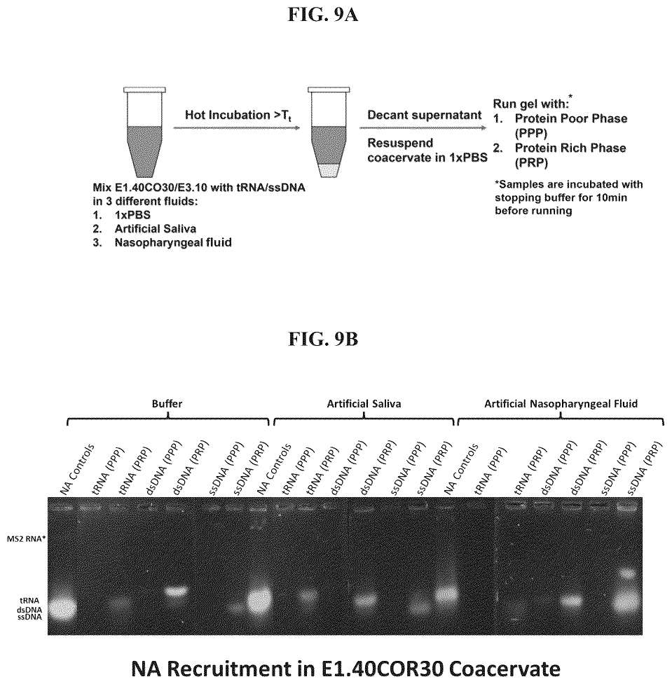

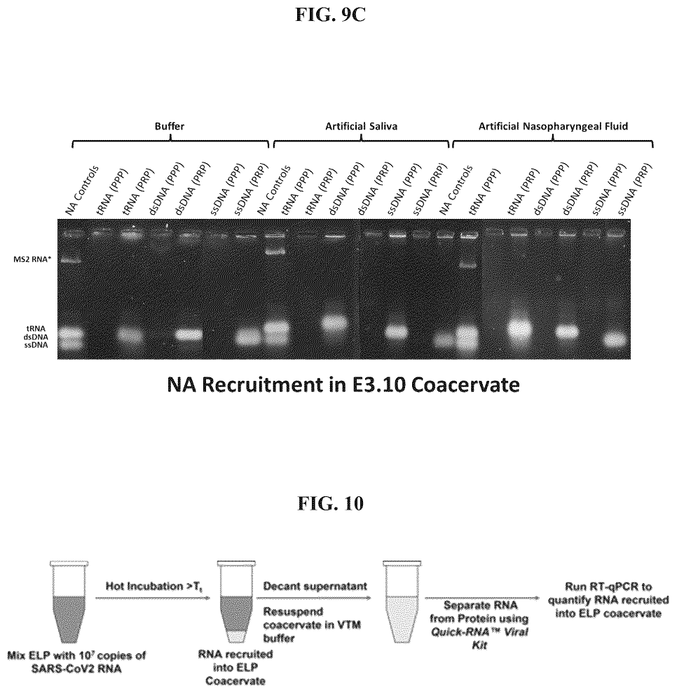

ELPs having an amino acid sequence of SEQ ID NOS. 5 and 6 can be used to extract nucleic acids (NA) into a protein poor phase (PPP) and a protein rich phase (PRP), as shown in FIG. 9A. SEQ ID NO. 5 is depicted as E1.40C030 and SEQ ID NO. 6 is depicted as E3.10. The PPP is relatively free of NA. The hot incubation step was performed at 55° C. The gels were run after breaking the complex of ELP and NA.

Solutions of ELPs and NAs in each physiologically relevant fluid were prepared, and the workflow of the experiment is described in FIG. 9A. The ELPs maintain their temperature-triggered phase behavior in artificial saliva and nasopharyngeal fluid. Qualitative examination of the presence of NAs in both the protein-rich and protein-poor liquid phases after ELP coacervation was performed by collecting samples of each phase, disrupting ELP-NA binding by dilution with a “stopping buffer”, and subjecting the solutes to NA gel electrophoresis.

Inspection of the agarose gels shown in FIGS. 9B and 9C suggests that ssDNA and tRNA are predominantly localized in ELP fusion protein-rich coacervate phases (lanes 3,5,8,10,13), indicating that, upon LLPS in all three media, both fusion proteins recruit tRNA and ssDNA into the condensed phase coacervates. Although RRM-RGG and HCV Core protein may bind different types of NA without any specific nucleotide preference, they tend to have some structural preference for complex structures such as hairpins or G-quadruplex. As shown, the structural properties of IDPs allow binding to NAs with complex structures.

### Example 4: Quantification of SARS-CoV-2 RNA in Complex Coacervates and Influence of ELP Fusions on qPCR

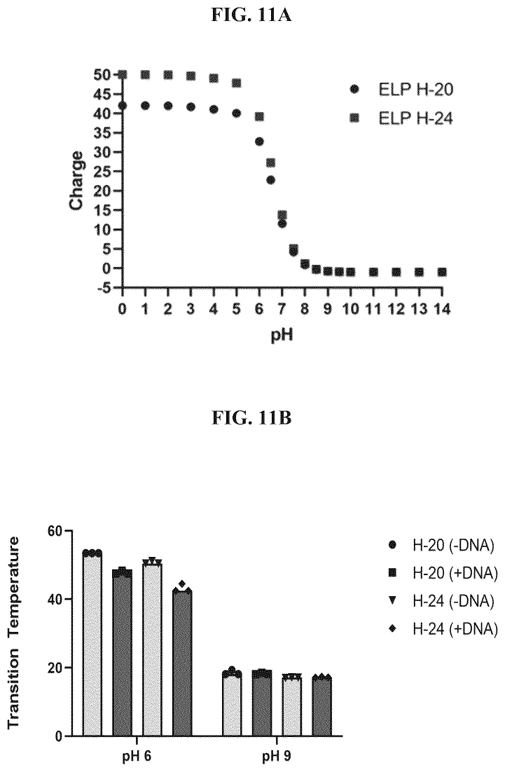

The binding and recruitment of viral RNA into ELP fusion condensates was determined for using NA-binding ELPs as reagents for RNA isolation from clinical samples for subsequent amplification and detection techniques such as PCR. Purified clinical SARS-CoV-2 RNA (1×10⁷ copies) was used as viral RNA for capture into ELP coacervates upon LLPS (FIG. 10). By comparison to a standard curve of dilutions of the SARS-CoV-2 RNA, the assay results confirmed that E3.10 and E1.40.COR30 could form NP condensates with viral RNA upon phase separation (Table 4 below). A mixture of 0.5 mM E3 and 10 μM E3.10 (which induces a more complete LLPS than E3.10 alone (i.e., complete phase coalescence) sequesters, and concentrates almost 105 copies of viral RNA from solution, while 0.5 mM E1.40.COR40 concentrates almost 106 viral RNA copies. The ELP fusion protein-RNA interactions can be deactivated to reduce interference of the ELPs in the RT-qPCR amplification process.

TABLE 4
E3.10 and E1.40.COR30 formation of NP condensates
with viral RNA upon phase separation

| Control Samples | CovRNA Copies | Log RNA | Ct |
|---|---|---|---|
| SARS-CoV-2 RNA | 100000 | 5 | 23 |
| | 10000 | 4 | 26 |
| | 1000 | 3 | 30 |
| | 100 | 2 | 35 |

| Sample | Ct | CovRNA Copies* |
|---|---|---|
| E3** + E3-10 Coacervate | 24 | 10^{4.7} |
| E1-40 COR30 Coacervate | 19 | 10^{5.9} |
| E3 + E3-10 Protein poor phase | Undetermined | Undetermined |
| E1-40 COR30 Protein poor phase | Undetermined | Undetermined |

*estimated amount of SARS-CoV-2 RNA copies recruited in the ELP coacervate upon LLPS
**E3 helps with LLPS

*estimated amount of SARS-CoV-2 RNA copies recruited in the ELP coacervate upon LLPS **E3 helps with LLPS

### Example 5: Dual Stimulus Response of IDPs with Temperature and pH

In some cases, IDPs can have an amino acid sequence of SEQ. ID. NO.: 7:

MSKGPG[XGVPG]_{L = 40,60,100,120}WP

wherein the ratio of X=V:H:G:A [1:2:1:1]. The conjugate acid (protonated form) of the imidazole side chain in histidine has a pKa of approximately 6.0. Thus, below a pH of 6, the imidazole ring is mostly protonated. The histidine content of the various lengths (L) of SEQ. ID. NO.: 7 results in a polycationic ELP and are as follows:
L=40:16
L=60:24
L=100:40
L=120:48

The phase of the polycationic ELP can be switchable according to pH. In some cases, a method for nucleic acid extraction with the ELP of SEQ. ID. NO.: 7, can include the following steps:
1. Mix
2. Heat shock
3. LLPS, decant supernatant
4. pH shift
5. LLPS, decant product

In two specific examples of the histidine ELPS of SEQ. ID. NO.: 7, two peptides were made. The amino acid sequence of SEQ. ID. NO.: 8 is also referred to herein as “H-20.” The amino acid sequence of SEQ. ID. NO.: 9 is also referred to herein as “H-24.”

SEQ. ID. NO.: 8:
MGH-[GVGVP GHGVP GGGVP GHGVP GAGVP]₂₀-GW
SEQ. ID. NO.: 9:
MGH-[GVGVP GHGVP GGGVP GHGVP GAGVP]₂₄-GW

The ELP can have a histidine in the “X” position of the pentapeptide of SEQ. ID. NO.: 2, at a position external to the VPGXG pentapeptide, or a combination thereof. For example, the ELP can have an amino acid sequence of SEQ. ID. NO. 10: [(VPGXG)₁₀-GHG]₈.

The electrostatic nucleic acid binding is expected to be modulated upon pH change, as shown in FIG. 11A. The charge on H-20 and H24 is shown as a function of pH as estimated by SnapGene Viewer (SnapGene software; snapgene.com). FIG. 11B depicts the measurement of Tt at pH 6 and pH 9 as measured by turbidimetry. At pH 6, where it is expected that each His-ELP will have ˜30-40 positive charges, a significant decrease is observed in the Tt for H-20 and H-24 in the presence of a ssDNA (FIG. 11B). Tt is not altered by the presence of ssDNA at pH 9, where it is expected that the His-ELPs would be close to neutral. As observed previously, NAs can alter the phase behavior of NA-binding ELPs. This suggests that at pH 6, the His-ELPs bind the ssDNA, while at pH 9, they do not. This pH-responsive binding behavior may be exploited to enable extraction of NAs upon LLPS. That is, aqueous solution conditions can be changed to weaken or strengthen the association between negatively-charged molecules, such as DNA or RNA, and His-ELPs, to create an on/off switch for protein-NA interactions. Thus, simple mechanisms of pH-dependent NA-binding and temperature and pH dependent LLPS of His-ELPs can be used in the task of NA isolation.

### Example 6: Extraction of SARS-CoV-2 RNA from COVID Patient Samples Using H-24 ELP

An ELP was created with an amino acid sequence of SEQ. ID. NO. 9, shown below:

SEQ. ID. NO.: 9:
MGH-[GVGVP GHGVP GGGVP GHGVP GAGVP]₂₄-GW

(shown as “H-24”). H-24 was applied in a two-step extraction process to isolate viral RNA from anonymized nasal swab samples from human patients that had previously been classified as either COVID positive ((+) SARS-CoV-2) or negative (“(−) SARS-CoV-2”) by a CDC-certified diagnostic method. The efficacy of an unoptimized His-ELP enabled extraction process was compared to a widely used commercial RNA extraction method (Quick-RNA™ Viral Kit, Zymos Research) in the detection of SARS-CoV-2 RNA by RT-qPCR.

The nasopharyngeal swab samples (suspended in VTM and DNA/RNA Shield™) were subjected to cell lysis by heat shock and then mixed the lysate with ELP H-24 in a pH 6 solution. The samples were incubated above the Tt of H-24 to induce LLPS with the objective of recruiting RNA into the protein coacervate phase to isolate it from the other lysate components. After phase separation, the protein-poor supernatant was pipetted out and the coacervate was resuspended in a pH 8.5 solution to disrupt electrostatic interactions between RNA and H-24. The solution was incubated above the Tt of the neutralized H-24 to induce LLPS with the objective of separating the H-24 protein from the RNA. The supernatant was pipetted out for PCR detection. Finally, the supernatants were diluted in nuclease-free water, as final extraction products are usually eluted in water.

The efficacy of His-ELP enabled extraction process was compared to that of a commercially available spin column methodology (Quick-RNA™ Viral Kit, Zymos Research) in the detection of SARS-CoV-2 RNA by RT-qPCR. The N1 primer/probe set used in the RT-qPCR experiments specifically amplifies a portion of the SARS-CoV-2 genome. Results of extraction of nasopharyngeal swab samples from a de-identified human COVID-19-positive patient were compared with those from a healthy human volunteer. Representative data from replicate measurements of each sample were made on different days and are provided below in Table 5. The primer/probe for PCR amplification of SARS-CoV-2 virus can be designed for any suitable unique region of the viral genome. Such primers and probes have been the subject of previous studies, including Anantharajah, A. et. al., How to choose the right real-time PCR primer sets for the SARS CoV-2 genome detection?, J. Vir. Met., 295 (2021) 114197.

Using the commercially available RNA extraction kit, SARS-CoV-2 RNA was detected in the samples from the COVID-19+ patient by RT-qPCR, with a cycle threshold (CT) value of 25. The CT value refers to the number of cycles necessary for the RT-qPCR process to detect a specific RNA sequence. A smaller value is correlated with higher RNA concentration in a sample. No SARS-CoV-2 RNA was detected in the sample from the healthy volunteer using the commercial RNA extraction kit. By comparison, after the two-step LLPS/pH switch process described above, RT-PCR did not detect SARS-CoV-2 RNA directly in the supernatant after the LLPS at pH 8.5, but it did detect it (CT=37) after the supernatant was diluted 1:50 with nuclease-free water. Interestingly, the target RNA was not detected after a similar 1:10, 1:20, nor 1:100 dilution, suggesting an optimal dilution, which may represent a balance of dilution of PCR inhibitors and sufficient SARS-CoV-2 RNA concentration for detection. The higher CT value obtained for the LLPS-based extraction suggests lower efficiency than the conventional extraction.

To examine whether the initial LLPS step at pH 6 resulted in incomplete capture of SARS-CoV-2 RNA into the coacervate phase, RT-qPCR was conducted on the supernatant obtained from that initial LLPS step. While detection of the target RNA was not possible directly in the supernatant, RNA was detected when this supernatant was diluted with nuclease-free water (1:10 CT=39; 1:20 CT=37; 1:50 CT=36; 1:100 not detected), with optimal detection (lowest CT) at 1:50 dilution. No SARS-CoV-2 RNA was detected in the sample from the healthy volunteer using the LLPS-based extraction under all dilution conditions studied.

These results demonstrate that the two-step H-24 LLPS process with pH shift is a simple process that is capable of extracting SARS-CoV-2 RNA from patient samples that is detectable by RT-qPCR, albeit after significant dilution in nuclease-free water and at higher CT than the standard commercial method. The amount of H-24 used in the extraction process, the time to achieve LLPS, and the solution conditions for LLPS in general, can be optimized. Also, alternative methods of lysis such as enzymatic lysis (eg. lysozyme), bead beating, or chemical lysis can be used instead of, or in addition to, the simple lysis procedure used here (heat shock).

TABLE 5

SARS-CoV-2 NP

| | (−) SARS-CoV-2 | (+) SARS-CoV-2 |
|---|---|---|
| Sample | C_{T} (RT-qPCR) | C_{T} (RT-qPCR) |
| Isolated RNA | Not Detected | 25 |
| [symbol] raditional Extraction) | | |
| SN 1 | Not Detected | Not Detected |
| SN 1 (1:10) | Not Detected | 39 |
| SN 1 (1:20) | Not Detected | 37 |
| SN 1 (1:50) | Not Detected | 36 |
| SN 1 (1:100) | Not Detected | Not Detected |
| SN 2 | Not Detected | Not Detected |
| SN 2 (1:10) | Not Detected | Not Detected |
| SN 2 (1:20) | Not Detected | Not Detected |
| SN 2 (1:50) | Not Detected | 37 |
| SN 2 (1:100) | Not Detected | Not Detected |

[symbol]

indicates data missing or illegible when filed

### Example 7. Electrostatic Binding Activity of his-ELPs to NAs at pH 6

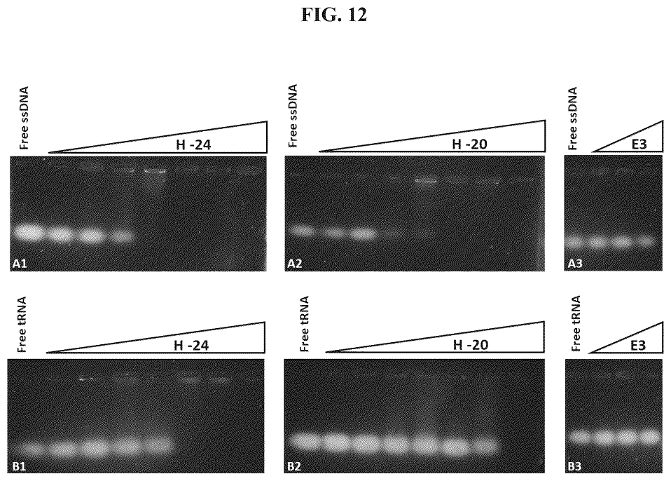

According to the prediction of His-ELP charge as a function of pH (FIG. 1l A), at pH 6, H-20 (SEQ. ID. NO. 8) is expected to have a charge of ≈+33 and H-24 (SEQ. ID. NO. 9) is expected to have a greater charge of ≈+39 as it has more histidines in its sequence. The primary association between His-ELPs and NAs is via electrostatic interactions; thus, the protein with greater charge is expected to have a more robust binding activity. Gel retardation assays were performed to analyze the binding activity of the His-ELPs (and another ELP with 8 cationic lysine charges, E3) with tRNA and ssDNA as a function of protein concentration at pH 6 (FIG. 12). Binding and gel electrophoresis were conducted at room temperature, below the Tt of each of the proteins, and for both tRNA and ssDNA, the degree of NA migration retardation was dependent on protein concentration (see FIG. 4). Moreover, the more charged protein polymer, H-24, retards migration of both NA species at lower concentrations, demonstrating that the His-ELP with higher charge exhibits a stronger binding. E3, which has only eight positively charged lysines, does not appreciably bind nucleic acids below its Tt (FIG. 4). As such, at pH 6, both His-ELPs exhibit NA-binding activity in their soluble state.

### Example 8: Reduced Electrostatic Binding of his-ELPs to NAs at Higher pH

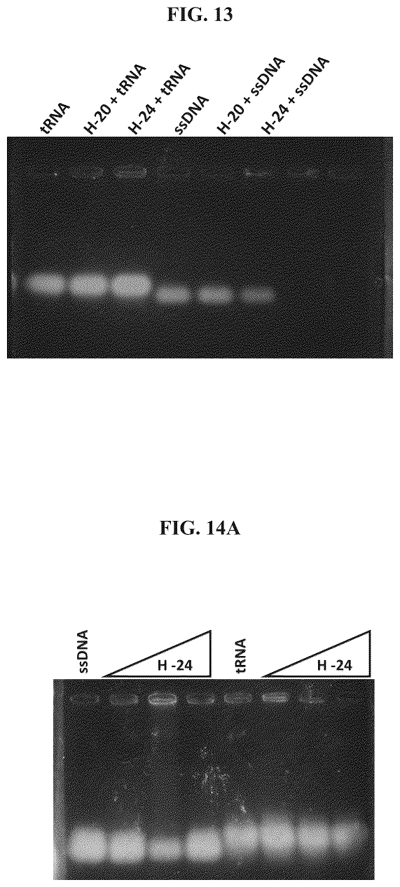

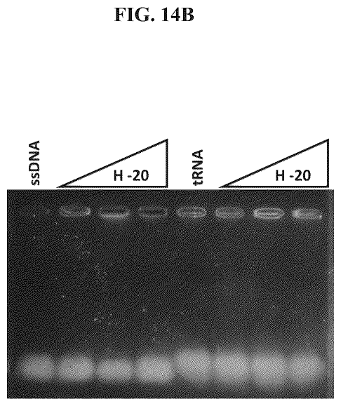

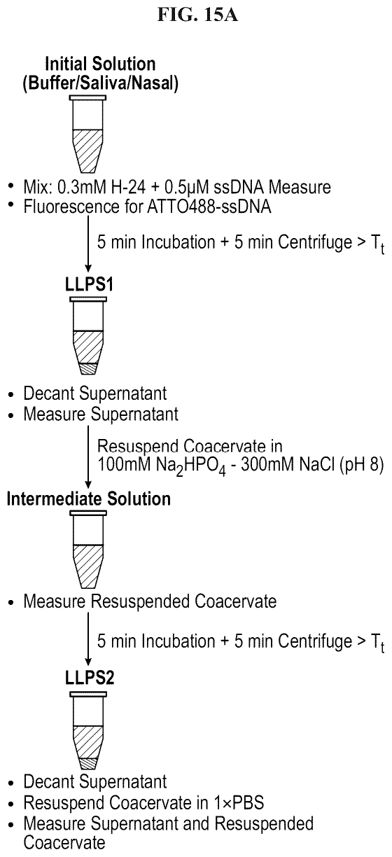

To demonstrate the modulation of electrostatic binding of His-ELPs and NAs, gel retardation assays were performed with mixtures of His-ELPs and NAs at pH 9, where the proteins are predicted to be slightly negatively charged (FIG. 13). To maintain the His-ELPs in their soluble state (below Tt), binding and gel electrophoresis were conducted at 4° C. The migration of tRNA and ssDNA was unaltered by the presence of His-ELPs at pH 9, confirming that in a higher pH environment, the neutralization of positive charges on the soluble His-ELPs (i.e., below their Tt) decreases their association with NAs (FIG. 13). A pH-induced change in charge can thus potentially be used as an on/off switch for His-ELP-NA interactions. However, pH 9 is not ideal for the stability of RNA, since exposure of RNA to highly alkaline solutions can lead to their degradation (hydrolysis). Consequently, a less basic buffered solution at pH 8 was employed to suppress associations between the His-ELPs and NAs (FIGS. 14A-B). At pH 8, the His-ELPs are predicted to have a slight positive charge. Binding in a buffer at pH 8 with added salt (300 mM NaCl) was evaluated for the following reasons: (i) NaCl will shield residual positive charges of the His-ELPs, and (ii) for isolation/extraction methods in coacervates that rely on the LLPS of ELPs, the addition of salt will reduce the transition temperature of His-ELP. A gel retardation assay showed that under these buffer and salt conditions, the migration of ssDNA and tRNA was not affected by the presence of soluble H-24 (FIG. 14A) and H-20 (FIG. 14B).

### Example 9: A Two-Step Separation Method Using Electrostatic Nucleic Acid Binding ELP to Isolate ssDNA from 3 Different Media Upon Two Subsequent LLPS

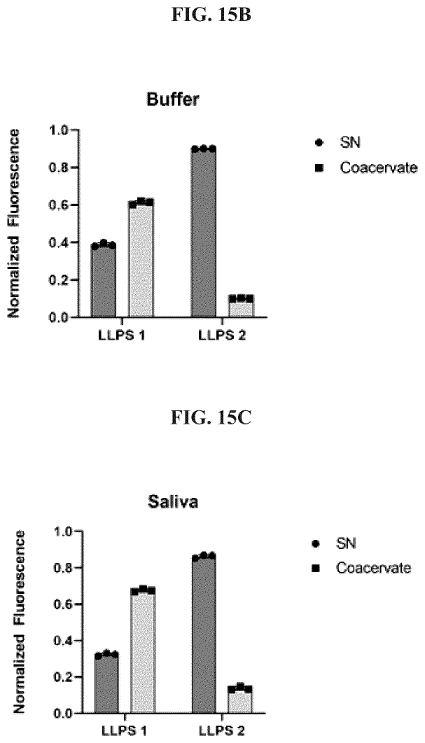

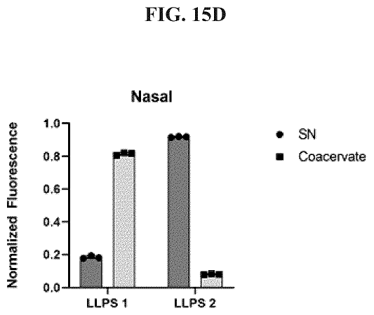

In a two-step nucleic acid separation method, pH can be adjusted to provide the ELP and nucleic acids in a sample with binding conditions in a first step, followed by separation conditions to release the nucleic acids from the ELP in a second step. For example, FIG. 15A shows a first step of making an initial solution of buffer (Citric Acid/Na₂HPO₄) at pH 6.5, a sample containing saliva and a nasopharyngeal swab which includes ssDNA, and an ELP of SEQ. ID. NO.: 9 (shown as “H-24”). The initial solution forms an LLPS1 (liquid-liquid phase separation no. 1) in which the bottom layer contains the ELP/ssDNA coacervate. In a second step, the pH is adjusted from 6.5 to 8.0, which results in release of the ssDNA from the ELP. The free ssDNA is found in the supernatant of LLPS2 (liquid-liquid phase separation no. 2), which can then be easily decanted.

FIGS. 15B-D graphically illustrate the fluorescence of the fluorescent label-tagged ssDNA in the samples shown in FIG. 15A. The fluorescence of the supernatant (SN) and coacervate of LLPS1 (at pH 6.5) and LLPS2 (at pH 8.0) for three different samples are shown. FIG. 15B shows the sample containing buffer with ssDNA. FIG. 15C shows the sample containing artificial saliva with ssDNA. FIG. 15D shows the sample containing artificial nasopharyngeal swab with ssDNA. In each of the samples, the ssDNA is found primarily in the coacervate in LLPS1 under ELP/ssDNA binding conditions at pH 6.5. In LLPS2, the ssDNA is found primarily in the supernatant in LLPS2 under ELP/ssDNA separating conditions at pH 8.0.

The specific compositions and methods described herein are representative, exemplary and not intended as limitations on the scope of the technology. Other objects, aspects, and embodiments will occur to those skilled in the art upon consideration of this specification and are encompassed within the spirit of the technology as defined by the scope of the claims. It will be readily apparent to one skilled in the art that varying substitutions and modifications may be made to the technology disclosed herein without departing from the scope and spirit of the technology. The terms and expressions that have been employed are used as terms of description and not of limitation, and there is no intent in the use of such terms and expressions to exclude any equivalent of the features shown and described or portions thereof, but it is recognized that various modifications are possible within the scope of the technology as claimed. Thus, it will be understood that although the present technology has been specifically disclosed by embodiments and optional features, modification and variation of the concepts herein disclosed may be resorted to by those skilled in the art, and that such modifications and variations are considered to be within the scope of this technology as defined by the appended claims and statements of the technology.

The technology illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The technology has been described broadly and generically herein. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein. In addition, where features or aspects of the technology are described in terms of Markush groups, those skilled in the art will recognize that the technology is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed is:

1. A method for isolating nucleic acids from a sample comprising nucleic acids, the method comprising:
(a) incubating the sample containing the nucleic acids with a population of polymers that bind the nucleic acids and form a coacervate in a liquid-liquid phase separated (LLPS) solution,
wherein the incubating is at a temperature above a concentration dependent phase separation transition temperature of the polymers;
(b) decanting the coacervate from the LLPS solution; and
(c) separating the nucleic acids from the polymers by adding a salt solution, adjusting the pH, or both to the LLPS solution to disrupt an electrostatic interaction between the nucleic acids and the polymers.

2. The method of claim 1, wherein the polymers comprise intrinsically disordered proteins.

3. The method of claim 2, wherein the intrinsically disordered proteins comprise polycationic elastin-like polypeptides, collagen, elastins, resilins, RRM-RGG and HCV Core proteins, or a combination thereof.

4. The method of claim 1, further comprising isolating the nucleic acids in the LLPS comprising the separated nucleic acids and polymers by centrifuging the LLPS and removing the supernatant or the coacervate, wherein the supernatant comprises the nucleic acids.

5. The method of claim 1, wherein the sample comprises a physiologically relevant sample or an environmental sample.

6. The method of claim 5, wherein the physiologically relevant sample comprises body tissue or body fluids.

7. The method of claim 4, wherein the physiologically relevant sample comprises saliva, sputum, mucus, nasopharyngeal discharge, blood, serum, plasma, urine, aspirate, stool or a combination thereof.

8. The method of claim 1, wherein the coacervate is in a protein rich phase of the LLPS.

9. The method of claim 3, further comprising separating the nucleic acids from the polycationic elastin-like polypeptide by adjusting the temperature above a transition temperature of the polycationic elastin-like polypeptide.

10. The method of claim 1, further comprising detecting the nucleic acids with a nucleic acid-based diagnostic assay.

11. The method of claim 10, wherein the nucleic acid-based diagnostic assay is a polymerase chain reaction (PCR) based assay or a non-PCR based assay.

12. The method of claim 10, wherein detecting the nucleic acids includes identifying viral RNA or DNA, quantifying viral RNA or DNA, or both.

13. The method of claim 1, wherein the polymer comprises a sequence segment with at least 95% sequence identity to any of SEQ. ID NOs: 1-10, wherein X in SEQ ID NO. 3 is any amino acid except proline.

14. The method of claim 3, wherein the polycationic elastin-like polypeptide has a sequence of SEQ. ID NO: 7, wherein the ratio of X to V:H:G:A is 1:2:1:1 and the phase of the polycationic elastin-like polypeptide is switchable from one phase to two phases by adjusting pH and temperature.

15. The method of claim 3, wherein the polycationic elastin-like polypeptide has histidine in the “X” position of SEQ. ID. NO.: 2, at a position external to a pentapeptide, or a combination thereof.

16. The method of claim 1, wherein the temperature above the concentration dependent phase separation transition temperature of the polymers is approximately 40-55° C.

17. The method of claim 3, wherein the polycationic elastin-like polypeptide has a sequence of SEQ. ID. NO. 9 and the incubating of the sample containing the nucleic acids with the polycationic elastin-like polypeptide to form the coacervate is performed under binding conditions of approximately pH 6.5.

18. The method of claim 17, wherein the pH is raised to approximately 8.0 to release the nucleic acids from the polycationic elastin-like polypeptide.

19. The method of claim 18, further comprising separating the nucleic acids from the polycationic elastin-like polypeptide by adjusting the temperature above a transition temperature of the polycationic elastin-like polypeptide.

20. A method for isolating nucleic acids from a sample comprising nucleic acids, the method comprising:
(a) incubating the sample containing the nucleic acids with a population of intrinsically disordered proteins to form a coacervate in a liquid-liquid phase separated solution, wherein the incubating is at a temperature above a concentration dependent phase separation transition temperature of the intrinsically disordered proteins;
(b) decanting the coacervate from the liquid-liquid phase separated solution; and
(c) separating the nucleic acids from the intrinsically disordered proteins by adding a salt solution, adjusting the pH, or both to the liquid-liquid phase separated solution to disrupt an electrostatic interaction between the nucleic acids and the intrinsically disordered proteins.

21. A composition comprising a polypeptide having a sequence of any of SEQ. ID NOs. 3 through 6, wherein amino acid X of the polypeptide of SEQ. ID. NO. 3 is any amino acid except proline.
